(19) United States
(12) Patent Application Publication
(10) Pub. No.: US 2014/0364500 A1
(43) Pub. Date: Dec. 11, 2014

(54) URSOLIC ACID SALTS FOR TREATING DIABETES AND OBESITY

(71) Applicants: Banavara L. Mylari, Lutz, FL (US); G. Alexander Fleming, Harpers Ferry, WV (US)

(72) Inventors: Banavara L. Mylari, Lutz, FL (US); G. Alexander Fleming, Harpers Ferry, WV (US)

(21) Appl. No.: 14/465,241

(22) Filed: Aug. 21, 2014

Related U.S. Application Data
(63) Continuation of application No. 13/864,509, filed on Apr. 17, 2013.
(60) Provisional application No. 61/672,351, filed on Jul. 17, 2012.

(51) Int. Cl. C07J 63/00; C07C 279/26

(52) U.S. Cl. CPC C07J 63/008; C07C 279/26
USPC 514/554; 562/498

(57) ABSTRACT

The present invention relates to compounds having the following structure:

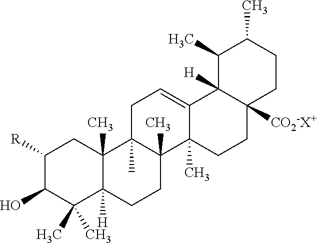

wherein R is H or OH and X⁺ is protonated metformin, protonated arginine, protonated lysine and protonated meglumine. The invention also relates to intermediates used in the preparation of such compounds, processes for the preparation of such compounds and intermediates, pharmaceutical compositions comprising such compounds and the methods of treatment using such compounds as antidiabetic, antiobesity, and antisarcopenia agents.

### CROSS-REFERENCE TO RELATED APPLICATIONS

This application claims priority of U.S. Provisional Patent Application Ser. No. 61/672,351, filed Jul. 17, 2012. This application is also a continuation of U.S. patent application Ser. No. 13/864,509, filed Apr. 17, 2013. The entire contents of both of these applications are incorporated herein by reference.

### BACKGROUND OF THE INVENTION

The present invention relates to ursolic acid and corosolic acid (2-α hydroxyl urosolic acid) salts of metformin, arginine, lysine, and meglumine, processes for preparing such salts, intermediates used in the preparation of such salts, processes for preparing such intermediates, pharmaceutical compositions comprising such salts and methods of treating diabetes and obesity in mammals comprising administering to said mammals said salts or said compositions.

Metformin, also known by other names including N,N-dimethylimidodicarbonimidic diamide and 1,1-dimethylbiguanide, is a known compound and it is disclosed in J. Chem. Soc., 1922, 121, 1790. The compound and its preparation and use are also disclosed, for example, in U.S. Pat. No. 3,174,901. Metformin is orally effective in the treatment of type 2 diabetes (T2D). Metformin is currently marketed in the United States in the form of its hydrochloride salt as an anti-hyperglycemic agent (formula I). Metformin hydrochloride can be purchased commercially and can also be prepared, for example, as disclosed in J. Chem. Soc., 1922, 121, 1790. It is postulated that metformin decreases hepatic glucose production and improves insulin sensitivity by increasing peripheral glucose uptake and utilization. Metformin hydrochloride is approved by the United States Food & Drug Administration for the therapeutic treatment of diabetes and it is widely regarded as the drug of choice for most patients with T2D.

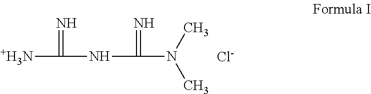

Age-related loss of skeletal muscle mass or sarcopenia results in decreased skeletal muscle strength, morbidity limitations, physical disability, and eventually high mortality among the elderly. Older adults with T2D with T2D have an altered body composition and low skeletal muscle strength compared non-diabetic older adults. Also, older diabetics with T2D loose their knee extensor strength more rapidly than non-diabetic counterpart. T2D is associated with excessive loss of skeletal muscle and trunk fat mass in the community dwelling older adults. Older women with type 2 diabetes are at especially high risk for loss of skeletal muscle mass (Park et al., Diabetes Care, published online Jun. 23, 2009).

Prediabetes is a syndrome. Many patients with type 2 diabetes and with a prediabetic condition known as metabolic syndrome suffer from a variety of lipid disorders including elevated triglycerides. The body uses triglycerides to store fat but high (>200 mg/dl) and very high (>500 mg/dl) triglycerides are associated with atherosclerosis which increases the patients risk of heart attack and stroke.

Incipient diabetes with impaired glucose tolerance is another prediabetic condition. Overall, type 2 diabetes and incipient diabetes with impaired glucose tolerance, are intimately intertwined with obesity, hyperlipidemia, including hypertriglyceridemia, and cardiovascular complications including arrhythmia, cardiomyopathy, myocardial infarction, stroke and heart failure. Clinically, pre-diabetes means that blood sugar level is higher than normal, but it's not yet increased enough to be classified as type 2 diabetes. Still, without intervention, prediabetes is likely to become type 2 diabetes over time

Obesity is associated with an increase in the overall amount of adipose tissue (i.e., body fat), especially adipose tissue localized in the abdominal area. Obesity has reached epidemic proportions in the United States. The prevalence of obesity has steadily increased over the years among all racial and ethnic groups. The most recent data from the Centers for Disease Control and Prevention, and the National Center for Health Statistics report 66% of the adult population overweight (BMI, 25.0-29.9), 31% obese (BMI, 30-39.9), and 5% extremely obese (BMI, >40.0). Among children aged 6 through 19 years, 32% were overweight and 17% were obese. This translates to 124 million Americans medically overweight, and 44 million of these deemed obese. Obesity is responsible for more than 300,000 deaths annually, and will soon overtake tobacco usage as the primary cause of preventable death in the United States. Obesity is a chronic disease that contributes directly to numerous dangerous co-morbidities, including type 2 diabetes, cardiovascular disease, inflammatory diseases, premature aging, and some forms of cancer. Type 2 diabetes, a serious and life-threatening disorder with growing prevalence in both adult and childhood populations, is currently the seventh of death in the United States. Since more than 80% of patients with Type 2 diabetes are overweight, obesity is the greatest risk factor for developing Type 2 diabetes. Increasing clinical evidence indicates that the best way to control Type 2 diabetes is to reduce weight.

Arginine and lysine are naturally occurring basic amino acids and meglumine is an amino sugar derived from sorbitol. All three of these in their protonated form (cf. Formulas III, IV, V, VI, respectively) are pharmaceutically acceptable for use as counter ions.

Ursolic acid and corosolic acid are naturally occurring plant substances and are members of the pentacyclic triterpene class of compounds. Frighetto et al. isolated ursolic acid as a major waxy, water-insoluble component of apple peals (Food Chemistry. 2008, 106, 767-771). The compounds have been shown to display a number of useful pharmacological properties including anti-inflammatory activity. Recently, urosolic acid is reported to show antiobesity and euglycemic efficacy in an obese mouse model. In another mouse model, ursolic acid is reported to reduce muscle atrophy and to stimulate muscle hypertrophy.

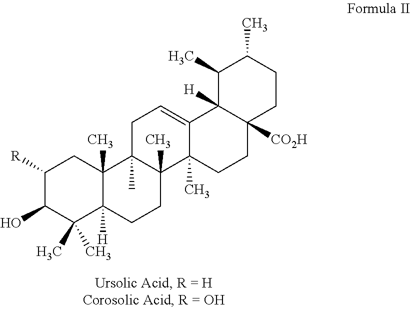

While ursolic acid and corosolic acid are insoluble in water, the salts of the present invention are more water soluble.

Water-soluble pentacyclic triterpene composition subjecting ursolic acid to inclusion in cyclodextrins is described by Sazuki et al in the U.S. Pat. No. 5,314,877 (May 24, 1994).

### SUMMARY OF THE INVENTION

The present invention relates to compounds of formulas III, IV, V and formula VI wherein R is H or OH, and X is protonated metformin, protonated arginine, protonated lysine, or protonated meglumine:

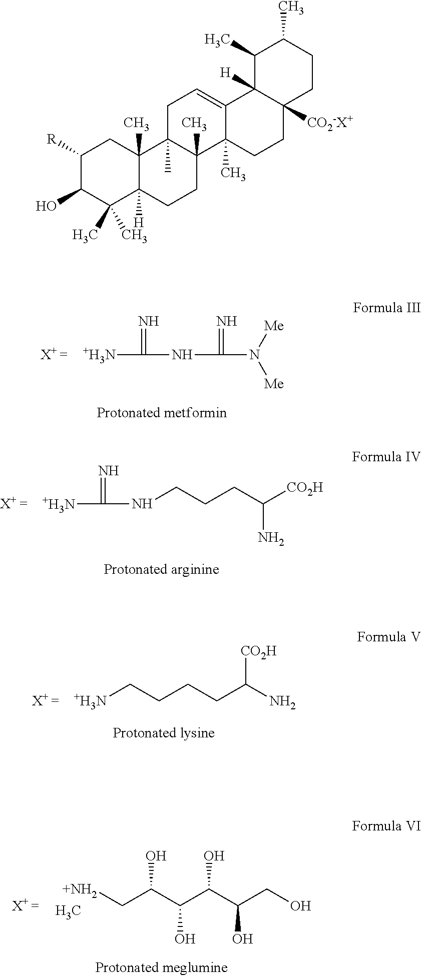

It should be understood that the location of the positive charge(s) in protonated metformin is illustrative only and it (they) could be located on other nitrogen atoms in metformin.

The compounds of the present invention include any polymorphs, solvates, and hydrates of the metformin salts described herein

The present invention also relates to a pharmaceutical composition comprising a salt of the present invention and a pharmaceutically acceptable carrier.

In one embodiment, the present invention relates to a pharmaceutical composition for the treatment of diabetes in mammals comprising an anti-diabetes effective amount of a ursolic acid or corosolic acid salt of the present invention and a pharmaceutically acceptable carrier. In one embodiment, the mammals are humans.

The present invention also relates to a method of treating diabetes in a mammal comprising administering to such mammal a ursolic acid or corosolic salt of the present invention. In one embodiment of the invention, the mammal is a human.

The present invention also relates to a method of treating diabetes in a mammal comprising administering to a mammal in need of such treatment an anti-diabetic effective amount of a ursolic acid or corosolic salt of the present invention. In one embodiment of the invention, the mammal is a human.

In one embodiment, the present invention relates to a pharmaceutical composition for the treatment of obesity in mammals comprising an anti-obesity effective amount of a ursolic acid or corosolic acid salt of the present invention and a pharmaceutically acceptable carrier. In one embodiment the mammals are humans.

The present invention also relates to a method of treating anti-obesity in a mammal comprising administering to the mammal a ursolic acid or corosolic salt of the present invention. In one embodiment of the invention, the mammal is a human.

The present invention also relates to a method of treating obesity in a mammal comprising administering to a mammal in need of such treatment an anti-diabetic effective amount of a ursolic acid or corosolic salt of the present invention. In one embodiment of the invention, the mammal is a human.

In a further embodiment, the present invention relates to a pharmaceutical composition for the treatment of sarcopenia in mammals comprising an anti-sarcopenia effective amount of a ursolic acid or corosolic acid salt of the present invention and a pharmaceutically acceptable carrier. In one embodiment, the mammals are humans.

The present invention also relates to a method of treating sarcopenia in a mammal comprising administering to the mammal a ursolic acid or corosolic salt of the present invention. In one embodiment of the invention, the mammal is a human.

The present invention also relates to a method of treating sarcopenia in a mammal comprising administering to a mammal in need of such treatment an anti-sarcopenia effective amount of a ursolic acid or corosolic salt of the present invention. In one embodiment of the invention, the mammal is a human.

One embodiment of the present invention relates to a unit dosage form for treatment of one of the foregoing diseases or conditions comprising an amount ursolic acid or corosolic salt of this invention effective to treat such disease or condition.

One embodiment of the present invention relates to a kit comprising a unit dosage comprising a ursolic acid or corosolic salt of this invention with instructions on how to use the kit and with provision for at least one container for holding the unit dosage form.

The terms “treating”, “treat”, or “treatment” as used herein include curative, preventive (e.g., prophylactic) and palliative treatment.

The present invention also relates to a process for preparing a compound of the formula XI by reacting a compound of formula VIII with salicylic acid (formula IX). The reaction herein is referred to as a coupling reaction.

### DETAILED DESCRIPTION OF THE PREFERRED EMBODIMENTS

The salts of the present invention include ursolic acid salt of metformin, corosolic acid salt of metformin, ursolic acid salt of arginine, ursolic acid salt of lysine, ursolic acid salt of meglumine, corosolic acid salt of metformin, corosolic acid salt of arginine, corosolic acid salt of lysine, and corosolic acid salt of meglumine.

One equivalent of metformin free base, prepared according the method of U.S. Pat. No. 3,957,853 (hereby incorporated herein by reference) may be dissolved in an appropriate reaction inert solvent. The solvent may be a polar solvent such as water. As used herein, the expression “reaction inert solvent” refers to a solvent or a mixture of solvents which do not interact with starting materials, reagents, intermediates or products in a manner which adversely affects the yield of the desired product. Preferred solvents include methanol, ethanol, n-propanol, isopropanol, acetone, ethyl methyl ketone, diethyl ketone and methyl isobutyl ketone. A particularly preferred solvent for this reaction is acetone. To this solution may be added a solution of one equivalent of ursolic acid or corosolic acid. One equivalent of metformin free base may be dissolved in an appropriate reaction inert solvent. The solvent may be a polar solvent such as water. As used herein, the expression “reaction inert solvent” refers to a solvent or a mixture of solvents which doesn't interact with starting materials, reagents, intermediates or products in a manner which adversely affects the yield of the desired product. Preferred solvents include methanol, ethanol, n-propanol, isopropanol, acetone, ethyl methyl ketone, diethyl ketone and methyl isobutyl ketone. A particularly preferred solvent for this reaction is acetone. To this solution may be added a solution of one equivalent of arginine, lysine or meglumine. The ursolic acid and corosolic acid salts of this invention can be isolated from the reaction mixture by methods well known to those skilled in the art, including according to the method set forth in U.S. Pat. No. 3,957,853, which is incorporated herein by reference, as are all of the other references cited herein.

The compounds of the present invention intended for pharmaceutical use may be administered alone or in combination with one or more other compounds of the invention or in combination with one or more other drugs (or as any combination thereof). Examples of such other drugs are antidiabetics (e.g., sulfonylureas, DPPIV inhibitors, SGLT 2 inhibitors) antihypertensives (e.g., ACE inhibitors, AR blockers, diuretics such as hydrochlorothiazide) and antihyperlipidemics (e.g., statins, fibrates, polyunsaturated acids such as eicosapentaenoic acid). Generally, the compounds of the present invention they will be administered as a formulation in association with a pharmaceutically acceptable carrier comprising one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995), which is incorporated herein by reference.

### Oral Administration

The compounds of the invention may be administered orally. Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

### Dosage

For administration to human patients, the total daily dose of the compounds of the invention is typically in the range 1 g to 12 g depending, of course, on the mode of administration. The condition being treated, and the age, sex and weight of the patient. In one embodiment the total daily dose is in the range 1 g to 10 g and in another embodiment the total daily dose is in the range 4 g to 8 g. The total daily dose may be administered in single or divided doses.

These dosages are based on an average human subject having a weight of about 65 kg to 70 kg. The physician will readily be able to determine doses for subjects whose weight falls outside this range, such as infants and the elderly.

The pharmaceutical composition may, for example, be in a form suitable for oral administration as a tablet, capsule, pill, powder, sustained release formulations, solution, or suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier and a compound according to the invention as an active ingredient. In addition, it may include other medicinal or pharmaceutical agents, carriers, adjuvants, etc.

Suitable pharmaceutical carriers include inert diluents or fillers, water and various organic solvents. The pharmaceutical compositions may, if desired, contain additional ingredients such as flavorings and binders.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Compounds of the formula III, IV, V and the formula VI can be tested for anti-diabetes activity as follows. Male Wistar rats, 8-10 weeks of age, 210-230 g. of body weight (bw) are used. The rats are housed at temperature of 18-21° C. on a 12 hour light-dark cycle. Rats are fed on a stock laboratory diet (59% carbohydrates, 17% protein, 3% fat, 21% minerals, water, and cellulose) and are allowed water ad libitum. Diabetes mellitus is induced in Wistar male rats by two intravenous injections of alloxan (40 mg/kg bw) in the tail vein. The rats are used in experiments 6 days after the first alloxan injection. Fasting glucose, insulin, total cholesterol, and triglycerides levels of these animals are recorded. Then rats are treated with metformin hydrochloride (100-300 mg/kg bw) for the next 5 days. On the sixth day, Fasting glucose, insulin, total cholesterol, and triglycerides levels of these animals are recorded.

Compounds of the formula III, IV, V and the formula VI can be tested for antiobesity activity and skeletal muscle strength activity in animal model, according the procedure described by Kunkel et al in Cell Metab. 2011, 13 (6), 627-638.

The following examples are meant to be illustrative of the practice of the invention, and not limiting in any way.

### Example 1

### Ursolic Acid Salt of Metformin

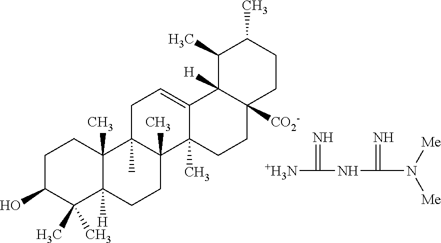

Ursolic acid salts metformin was prepared according to the scheme shown below.

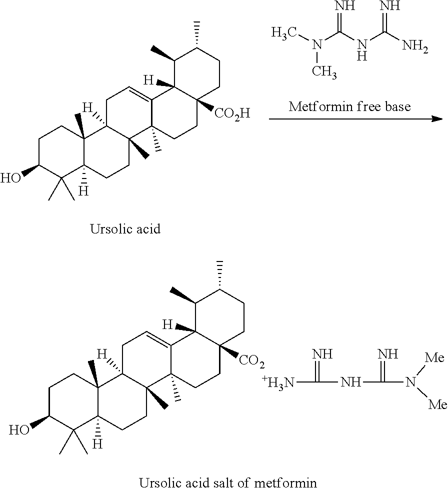

### Metformin Free Base.

N,N-dimethylimidodicarbonimidic diamide hydrochloride (metformin hydrochloride, 4.01 g, 24.3 mmol) was dissolved in 1N sodium hydroxide in water (24.2 mL, 24.2 mmol) and stirred at room temperature for 30 minutes. The solution was concentrated in vacuum and the white residue was taken up in 80 mL ethanol. The mixture was carefully concentrated to yield a white solid. The material was taken up in 60 mL ethanol and the solution was filtered to remove precipitated sodium chloride. The filtrate was concentrated to a white solid that was placed on high vacuum overnight to yield metformin free base (3.18 g, 102%) as a white solid.

### Ursolic Acid Metformin Salt.

Metformin free base (0.80 g, 6.2 mmol) was stirred in acetonitrile (30 mL) for 10 min. In a separate 200 mL round-bottom flask, ursolic acid (2.00 g, 4.38 mmol) was suspended in acetonitrile (100 mL). The metformin free base solution contained some precipitate (NaCl), so it was filtered through fluted filter paper into the ursolic acid suspension. The mixture was red stir 16 h. The white solid that formed was isolated by filtration and washed with acetonitrile (100 mL). The solid was dried via suction and placed under high vacuum at 50° C. for 4 h to remove any residual solvent. Ursolic acid metformin salt (2.42 g; yield, 94%) was isolated as a white powder. Melting point (uncorrected): 228-230° C. (decomposition). Elemental analysis: Calculated: C, 69.70%; H, 10.15%; N, 11.95%. Found: C, 69.52%; H, 10.25%; N, 11.86%. Water: 0.11% (Karl Fischer). ¹H NMR (300 MHz, DMSO-d₆) δ ppm 0.57-0.72 (m, 4H) 0.74-0.93 (m, 19H) 0.99 (s, 3H) 1.06-1.58 (m, 13H) 1.60-1.86 (m, 3H) 1.92-2.09 (m, 1H) 2.17 (d, J=11.46 Hz, 1H) 2.90 (s, 6H) 2.95-3.04 (m, 1H) 4.26 (br. s., 1H) 4.98 (br. s., 1H) 6.50-9.00 (br. s., 6H). ¹³C NMR (101 MHz, DMSO-d₆) δ ppm 15.26, 16.13, 17.24, 17.48, 18.09, 21.55, 22.87, 23.38, 24.77, 27.04, 28.07, 28.31, 31.15, 32.99, 36.59, 37.29, 38.28, 38.40, 41.76, 47.06, 47.29, 53.27, 54.89, 76.88, 122.79, 140.02, 158.21, 160.48, 180.59. MS (ESI+) for metformin C₄H₁₁N₅ m/z 130.1 (M+H)⁺, MS (ESI−) for ursolic acid C₃₀H₄₈O₃ m/z 455.3 (M−H)⁻. HPLC retention time: 5.868 min. HPLC conditions: Agilent 1100 HPLC: Eclipse XDB-C18 50×4.6 mm 1.8 micron column: Gradient—5 min 95% water (0.10% TFA) to 95% acetonitrile (0.07% TFA); 1.5 mL/min; UV Detection at 210 nM.

### Example 2

### Solubility Determination

The solubility of metformin ursolate in water was determined by an HPLC assay (conditions given below). Four known concentrations of metformin ursolate dissolved in acetonitrile were assayed by HPLC assay and standard linear regression was used to determine the equation for line of best fit. A saturated solution of metformin ursolate in water was assayed in triplicate by HPLC. The average AUC of the three runs was used to determine the concentration.

Known Concentrations (μg/mL): 41.7, 83.3, 166.6, and 333.2
Equation for line of best fit:
y=2.0049x−1.0893 (r²=0.9998)
y=concentration
x=absorbance

### HPLC Conditions:

Agilent 1100 HPLC; Eclipse XDB-C18 50×4.6 mm 1.8 micron column; Gradient—5 min 95% water (0.10% TFA) to 95% acetonitrile (0.07% TFA); 1.5 mL/min; UV Detection @ 210 nm.

The solubility of metformin ursolate in water was found to be 74 μg/mL.

Ursolic acid was not detected by HPLC assay when water saturated with ursotic acid was filtered and the filtrate was assayed as described above, confirming the literature citation that ursolic acid is insoluble in water (Merck Index, 11^{th} Edition, page 1556). So, metformin ursolate is much more soluble in water than is ursolic acid.

1-27. (canceled)

28. A compound having the structure: 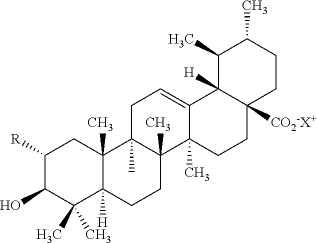
wherein R is H and X⁺ is protonated metformin.

29. A pharmaceutical composition comprising a unit dosage of a compound according to claim 28 and a pharmaceutically acceptable carrier.

30. A pharmaceutical composition according to claim 28 wherein the unit dosage of the compound is an antidiabetic effective amount.

31. A pharmaceutical composition according to claim 28 wherein the unit dosage of the compound is an antiobesity effective amount.

32. A pharmaceutical composition according to claim 28 wherein the unit dosage of the compound is an antisarcopenia effective amount.

33. A kit comprising: a) a unit dosage comprising the compound of claim 28; b) instructions on how to use the kit; and c) at least one container for holding the unit dosage forms.

34. A method of treating diabetes in a mammal, comprising administering to a mammal in need of such treatment an antidiabetic effective amount of a compound according to claim 28.

35. A method of treating obesity in a mammal, comprising administering to a mammal in need of such treatment an antiobesity effective amount of a compound according to claim 28.

36. A method of treating sarcopenia in a mammal, comprising administering to a mammal in need of such treatment an antisarcopenia effective amount of a compound according to claim 28.

37. A method of treating pre-diabetes in a mammal comprising administering to a mammal, including in need of such treatment a pre-antidiabetic effective amount of a compound according to claim 28.
